(19) United States
(12) Patent Application Publication
(10) Pub. No.: US 2020/0369997 A1
(43) Pub. Date: Nov. 26, 2020

(54) VITRO FERTILIZATION DEVICE AND METHOD FOR PROTECTING CELLS FROM LIGHT

(71) Applicants: Jozsef BODIS, Pecs (HU); Krisztina GODONY, Pecs (HU)

(72) Inventors: Jozsef BODIS, Pecs (HU); Krisztina GODONY, Pecs (HU)

(21) Appl. No.: 16/879,804

(22) Filed: May 21, 2020

Related U.S. Application Data
(60) Provisional application No. 62/850,631, filed on May 21, 2019.

(51) Int. Cl. C12M 1/22; C12M 1/32; C12M 1/00; C12M 3/00

(52) U.S. Cl. CPC C12M 23/10; C12M 23/12; C12M 21/06; C12M 23/38; C12M 41/10; C12M 23/34

(57) ABSTRACT

A device and method for protecting cells, gametes, embryos, and oocytes from the harmful effect of illumination. The device is a culture dish that is optimized to create a dark environment inside of the dish. The culture dish features a base, an exterior wall connected to the outer rim of the base that forms an enclosure, and a lid connected to the exterior wall. The dish is preferably made of polystyrene and lid is preferably made of aluminum foil.

The method features the following steps: reducing a room exposure to sunlight, where the cells and embryos are located; covering the room ceiling lighting with color filter-luminaires; shielding the transparent surfaces of the applied aspiration set and test tube from light; applying a colored red-light filter to the IVF workstation; and incubate cells and embryos in a light-protected place.

### FIELD OF INVENTION

The present invention pertains to the field of assisted reproductive technology, and in particular, to a device and method for protecting cells, gametes, embryos, and oocytes from the harmful effects of illumination.

### BACKGROUND OF THE INVENTION

In vitro fertilization (IVF) is a form of assisted reproductive technology (ART). IVF has become a routine medical procedure. During the course of IVF, gametes and embryos are being handled frequently. This handling implies physical handling, for example, moving the cells from one culture dish to another, denudation and Intracytoplasmic Sperm Injection (ICSI) and changes in medium composition and physical parameters such as temperature, pH, osmolality, and light exposure.

To improve the success rate of IVF, proper care must be taken to protect the cells and embryos during the procedure. Environmental factors, such as visible and non-visible light, might have a large influence on the functioning of cells under artificial laboratory circumstances.

Successful outcomes in IVF require optimal environmental conditions for gametes and embryos throughout the entire procedure, where they can be affected by stress factors during the culture procedure. Different stressors can amplify the overall effect of even low levels of damage. When embryos are exposed to a harmful environmental factor in a culture system with other stressors like ovarian stimulations, inappropriate culture conditions, light, there is additive effect thereby amplify impacting on an embryos' viability, which alone could have been relatively benign. Adverse effects can lead to reduced or abnormal implantation rates showing that damages can impact the embryos viability or potential to become a pregnancy. The risk of damage is real; thus, it is necessary to minimize all of the risks for potential damage associated with the whole in vitro fertilization procedure.

Therefore, what is needed is a device and method for protecting cells, gametes, embryos, and oocytes from the harmful effects of illumination.

### SUMMARY OF THE INVENTION

The present invention provides protection to cells, gametes, embryos, and oocytes from the harmful effect of illumination. According to at least some embodiments, a culture dish is optimized to create a dark environment inside of the dish, where dish features a base, an exterior wall connected to the outer rim of the base that forms an enclosure, and a lid connected to the exterior wall. The dish is preferably made of polystyrene and lid is preferably made of aluminum foil.

According to at least some embodiment, the method of protecting cells, gametes, embryos, and oocytes from light comprises the following steps: reducing a room exposure to sunlight, where the cells and embryos are located; covering the room ceiling lighting with color filter-luminaires; shielding the transparent surfaces of the applied aspiration set and test tube from light; applying a colored red-light filter to the IVF workstation; and incubate cells and embryos in a light-protected place.

### BRIEF DESCRIPTION OF THE DRAWINGS

These and other features, aspects, and advantages of the present invention will become better understood with regard to the following description, appended claims, and accompanying drawings where:

FIGS. 1A to 1D illustrate micro-droplet culture dishes;

FIGS. 2A to 2D illustrate 4/7-well culture dishes for ensuring embryo viability;

FIGS. 2C to 2D illustrate an alternative 4/7-well culture dish;

FIGS. 3A to 3B illustrate the center well dish;

FIGS. 4A to 4B illustrate a culture dishes covered with a lid;

FIGS. 5A to 5F illustrate culture dishes of different sizes for oocyte retrieval fertilization and culture;

FIG. 6A illustrates an embryo transfer catheter;

FIG. 6B illustrates the catheter hub; and

FIG. 7 illustrates the process of protecting cells, oocytes, and embryos from light.

### DETAILED DESCRIPTION

Many aspects of the present invention can be better understood with reference to the following drawings. While one implementation is described hereto, it is to be understood that other variations are possible without departing from the scope and nature of the present invention.

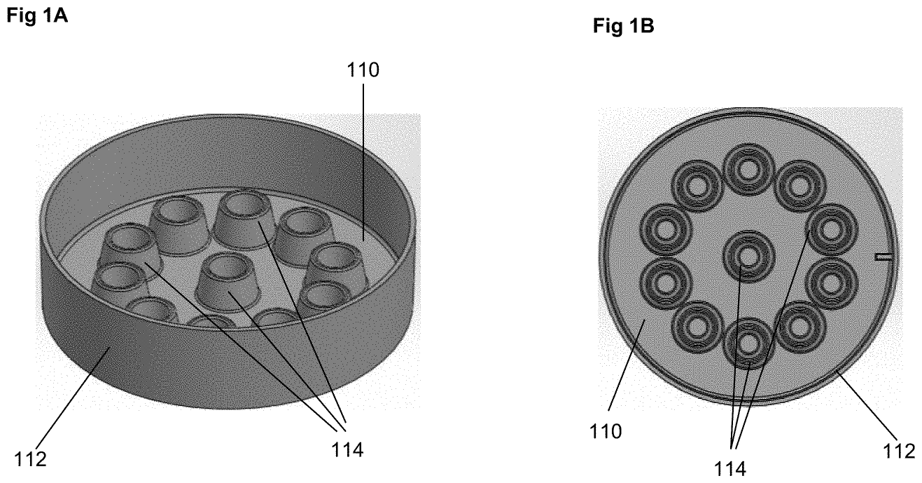

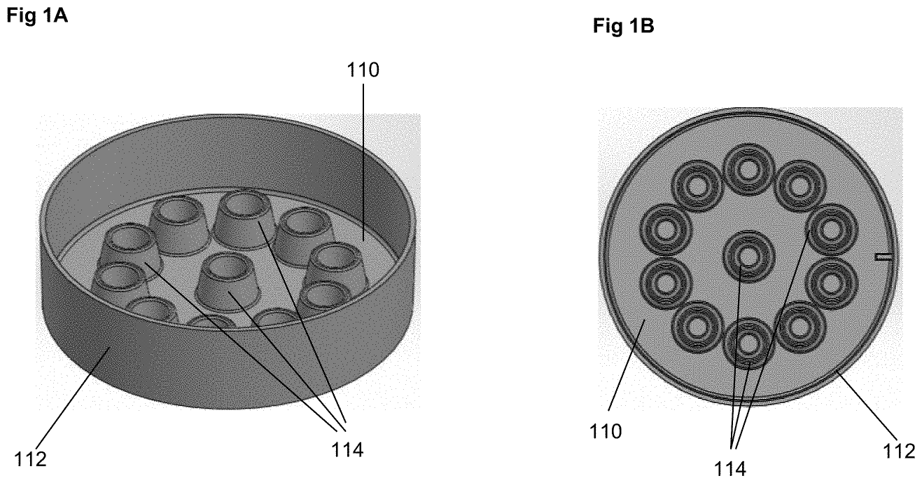

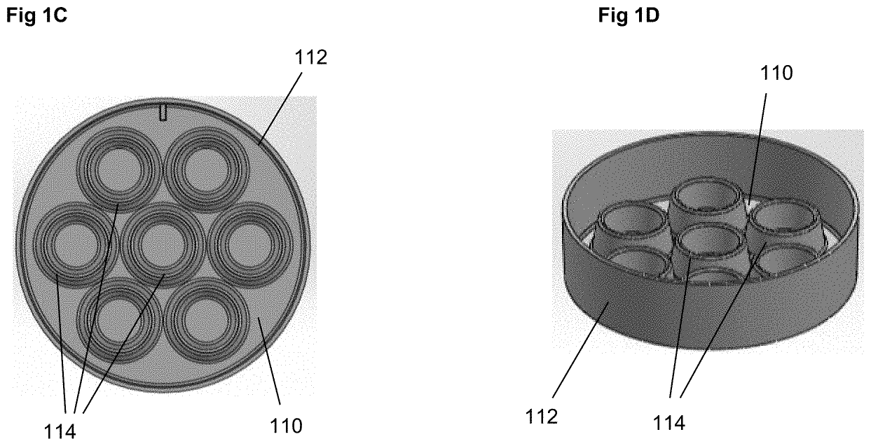

FIGS. 1A to 5F are examples of various types of culture dishes that can be covered or are covered with a lid to protect cells, gametes, embryos, and oocytes from the harmful effects of illumination.

FIGS. 1A to 1D illustrate micro-droplet culture dishes. The micro-droplet culture dish is preferably made of polystyrene and features a base 110 and an exterior wall 112 that is connected to the outer rim of the base 110 to form an enclosed base. The base 110 is circular with a thickness of 0.8 mm, preferably. The diameter of the base 110 can be either 40 mm, 58 mm, or 99 mm. However, the diameter of the base is not limited to the above lengths. Although circular base 110 is shown, other shapes may be used, such as a rectangular base.

A plurality of microwells 114 is attached to the base 110 inside of the enclosure. These microwells are 7/11 microwells 114, which are optimized for holding the range of 25 to 35 μL droplets.

Although not shown, the lid attaches to the exterior wall 112 to form an enclosed container with the base 110 and the exterior wall 112. The lid is made of a material that protects cells and embryos from light. The lid is able to rotate on its axis, which is parallel to the axis of the base 110. The thickness of the lid ranges from 0.8 mm to 1.00 mm. The lid is preferably made of a thin sheet of aluminum, such as aluminum foil. Although aluminum is used, other material can be used if the material has no toxic effect on embryos and is able to prevent the transmission of light to the embryos.

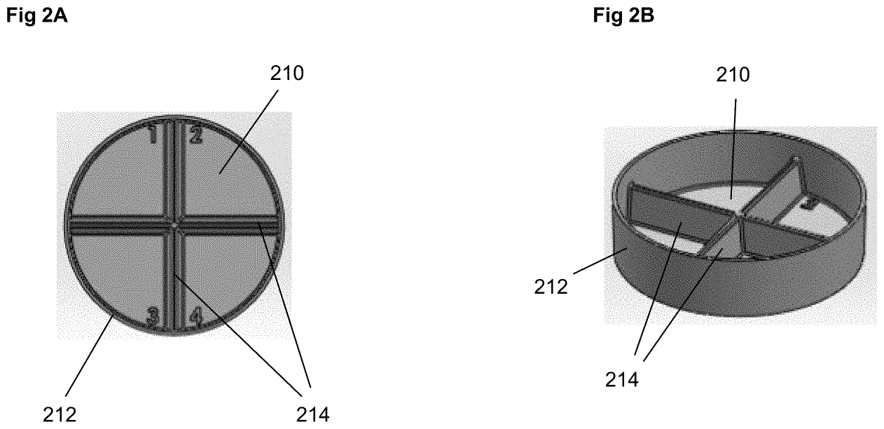

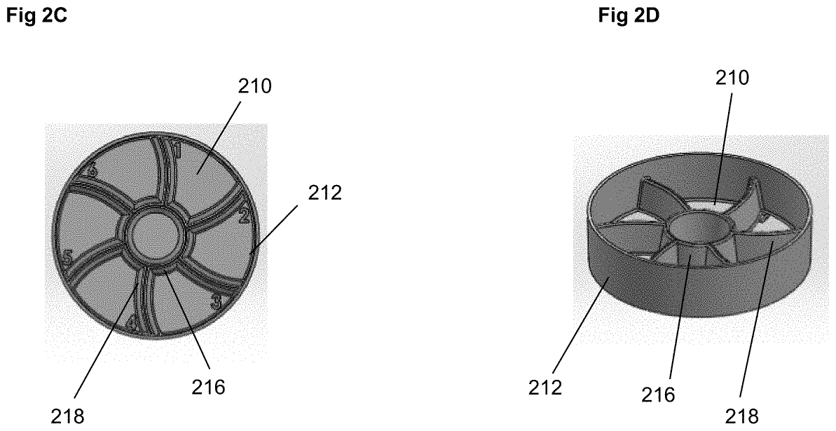

FIGS. 2A to 2D illustrate 4/7-well culture dishes for ensuring embryo viability. In FIGS. 2A to 2B, the 4/7-well culture dish is preferably made of polystyrene and features a base 210 and an exterior wall 212 that is connected to the outer rim of the base 210 to form an enclosed base. The base 210 is circular with a thickness of 0.8 mm, preferably. The diameter of the base 210 can be either 40 mm, 58 mm, or 99 mm. However, the diameter of the base is not limited to the above lengths. Although circular base 210 is shown, other shapes may be used, such as a rectangular base.

Two interior walls are located inside of the enclosure and are attached to the base 210 and the exterior wall 212. Each interior wall 214 is a diagonal of the base 210 and intersects perpendicularly with the other interior wall. The two interior walls 214 and the exterior wall 212 create four wells for housing material, such as cells. Either air or media, resulting in a homogenous temperature, surround each well.

Although not shown, the lid attaches to the exterior wall 212 to form an enclosed container with the base 210 and the exterior wall 212. The lid is made of a material that protects cells and embryos from light. The lid is able to rotate on its axis, which is parallel to the axis of the base 210. The thickness of the lid ranges from 0.8 mm to 1.00 mm. The lid is preferably made of a thin sheet of aluminum, such as aluminum foil.

FIGS. 2C to 2D illustrate an alternative 4/7-well culture dish. Similarly, this 4/7-well culture dish is preferably made of polystyrene and features a base 210 and an exterior wall 212 that is connected to the outer rim of the base 210 to form an enclosed base. The base 210 is circular with a thickness of 0.8 mm, preferably. The diameter of the base 210 can be either 40 mm, 58 mm, or 99 mm. However, the diameter of the base is not limited to the above lengths. Although circular base 210 is shown, other shapes may be used, such as a rectangular base.

A circular interior wall 216 is attached to the base 210 inside of the enclosure to form a center well. A plurality of curved interior walls 218 is connected to the circular interior wall and the exterior wall 212 to form six wells.

Although not shown, the lid attaches to the exterior wall 212 to form an enclosed container with the base 210 and the exterior wall 212. The lid is made of a material that protects cells and embryos from light. The lid is able to rotate on its axis, which is parallel to the axis of the base 210. The thickness of the lid ranges from 0.8 mm to 1.00 mm. The lid is preferably made of a thin sheet of aluminum, such as aluminum foil.

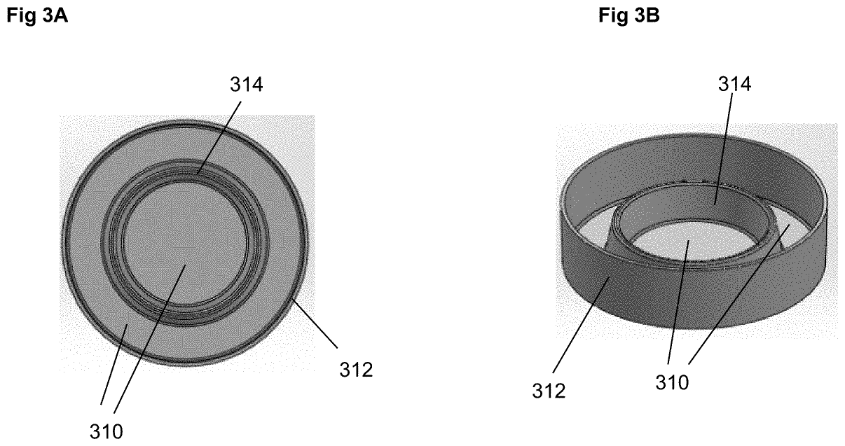

FIGS. 3A to 3B illustrate the center well dish. The center well dish is a multi-purpose dish for fertilization, cryo-procedures, embryo culture, and embryo transfer. The center well dish is preferably made of polystyrene and features a base 310 and an exterior wall 312 that is connected to the outer rim of the base 310 to form an enclosed base. The base 310 is circular with a thickness of 0.8 mm, preferably. The diameter of the base 310 can be either 40 mm, 58 mm, or 99 mm. However, the diameter of the base is not limited to the above lengths. Although circular base 310 is shown, other shapes may be used, such as a rectangular base.

A circular interior wall 314 is attached to the base 310 inside of the enclosure to form a center well. The center well allows for easy instrument access to the embryos sitting in the center well. The center well can be accessed only from the top to prevent harm or damage to the embryos from manipulation with devices.

Although not shown, the lid attaches to the exterior wall 312 to form an enclosed container with the base 310 and the exterior wall 312. The lid is made of a material that protects cells and embryos from light. The lid is able to rotate on its axis, which is parallel to the axis of the base 310. The thickness of the lid ranges from 0.8 mm to 1.00 mm. The lid is preferably made of a thin sheet of aluminum, such as aluminum foil.

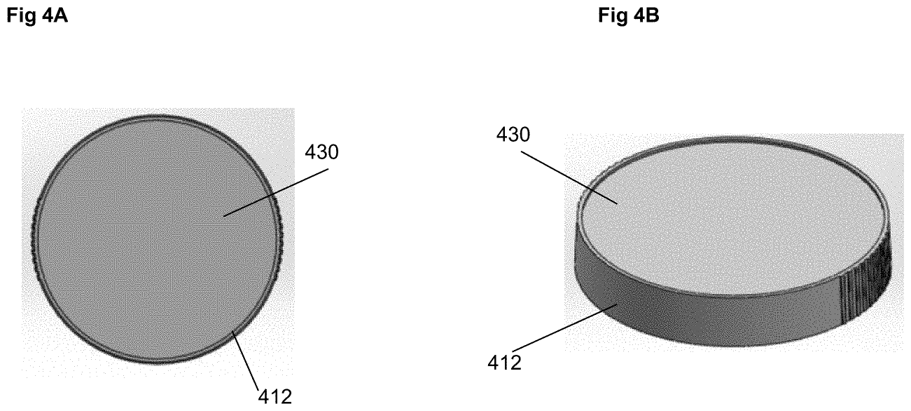

FIGS. 4A to 4B illustrate a culture dishes covered with a lid 430. Each culture dish has a base and exterior wall connected the base as the previous dishes discussed. The lid 430 is attached to the exterior wall 412. The lid 430 protects is made of a material that protects cells and embryos from light. The lid is able to rotate on its axis, which is parallel to the axis of the base. The thickness of the lid ranges from 0.8 mm to 1.00 mm. The lid is preferably made of a thin sheet of aluminum, such as aluminum foil.

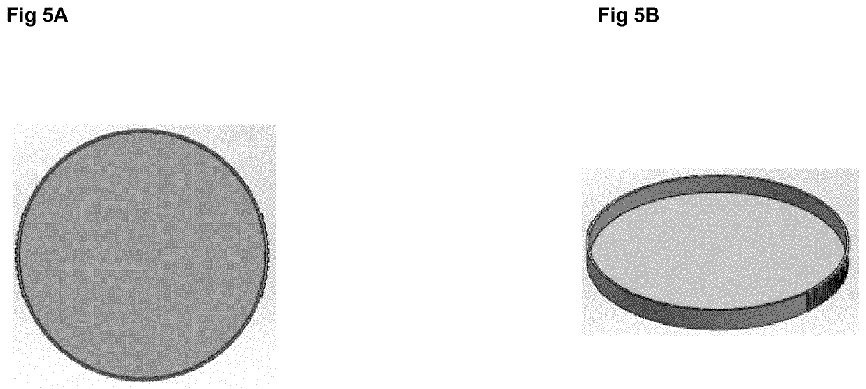

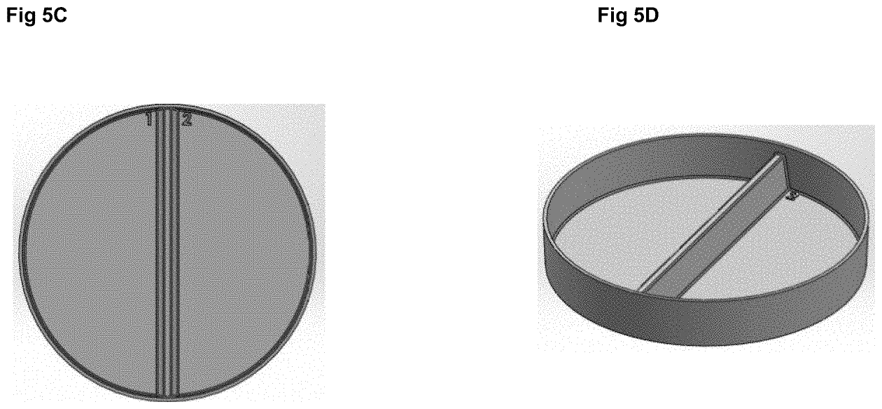

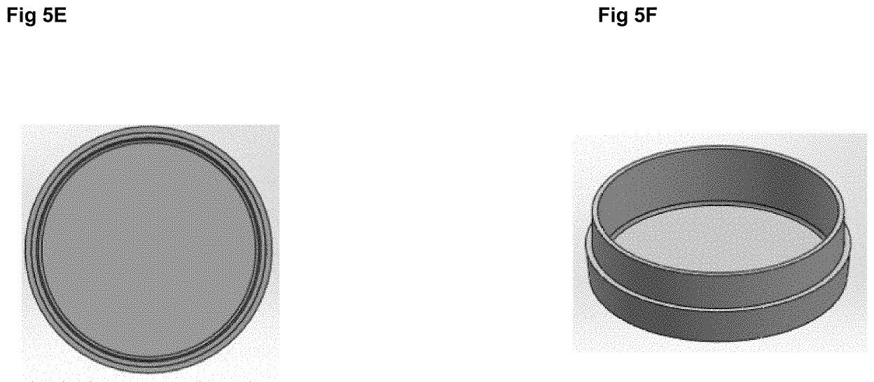

FIGS. 5A to 5F illustrate culture dishes of different sizes for oocyte retrieval fertilization and culture. For FIGS. 5A to 5F, each dish is preferably made of polystyrene and features a base and an exterior wall that is connected to the outer rim of the base to form an enclosed base. The base is circular with a thickness of 0.8 mm, preferably. The diameter of the base can be either 40 mm, 58 mm, or 99 mm. However, the diameter of the base is not limited to the above lengths. Although the circular base is shown, other shapes may be used, such as a rectangular base.

Although not shown, a lid attaches to the exterior wall to form an enclosed container with the base and the exterior wall. The lid is made of a material that protects cells and embryos from light. The lid is able to rotate on its axis, which is parallel to the axis of the base. The thickness of the lid ranges from 0.8 mm to 1.00 mm. The lid is preferably made of a thin sheet of aluminum, such as aluminum foil.

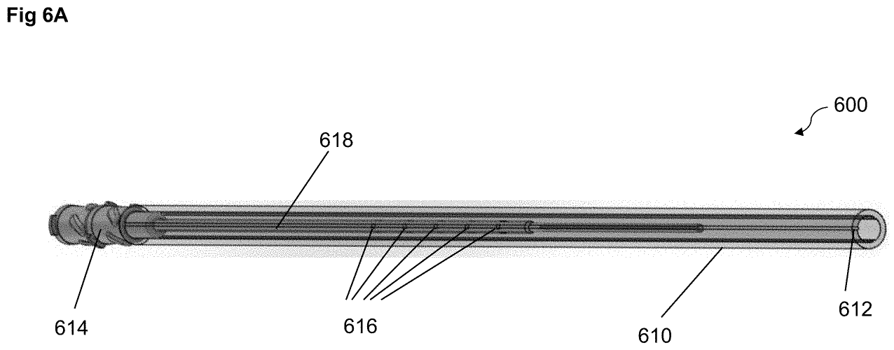

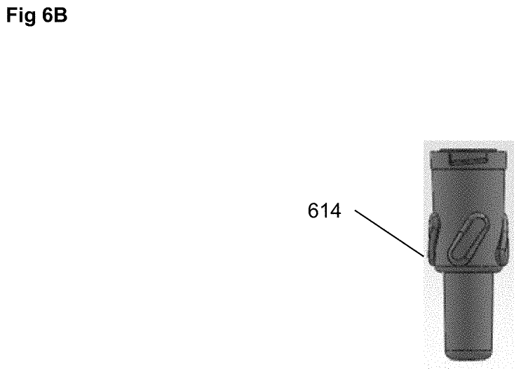

FIG. 6A illustrates an embryo transfer catheter. The catheter 610 features a formable outer sheath 610, a tip 612 located at one end of the formable outer sheath 610, and catheter hub 614 attached to the opposite end of the formable outer sheath 610.

The formable outer sheath 610 features an inner-centimeter markings 616 to show out sheath depth and an inner lumen protrusion 618 for the confidence of placement within the uterus. The catheter 610 is made preferably from silicon, which prevents the light exposure. Other suitable material that prevents light exposure may be used.

The length and diameter of the catheter may vary, but must allow the transfer of cells, oocytes, and embryos. For example, a catheter with a diameter of 0.5 mm and length of 234 mm would allow the transfer.

FIG. 6B illustrates the catheter hub 614. The catheter hub 614 connects the catheter 610 to other medical devices. As shown, the catheter hub 614 is comprised of an upper and lower part. The upper part has a height of 10.30 mm and an outer diameter of 4.20 mm. The lower part has a height of 7.3 mm and diameter of 1.4 mm. The dimensions of the catheter hub are not restricted to the above example and may vary.

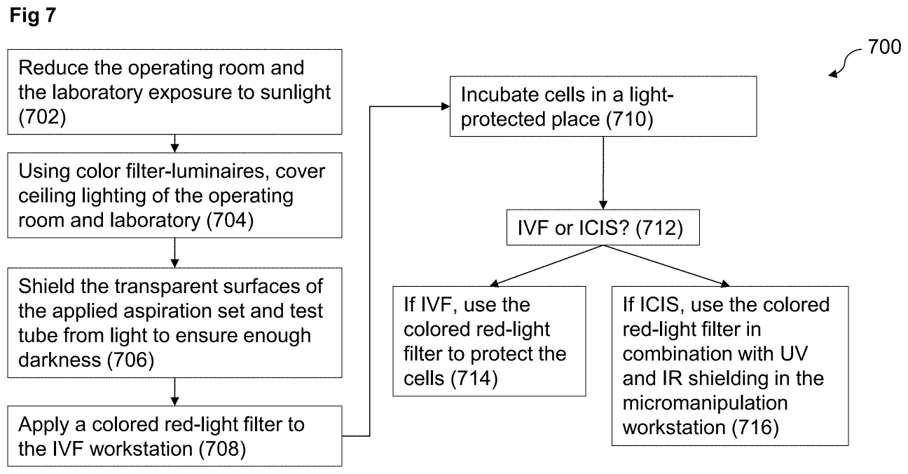

FIG. 7 illustrates the process of protecting cells, oocytes, and embryos from light.

The process 700 starts with reducing the operating room and the laboratory exposure to sunlight (Step 702). In addition, the ceiling lighting of the operating room and laboratory is covered with color filter-luminaire (Step 704). Next, shielding is applied to transparent surfaces of the applied aspiration set and a test tube to ensure enough darkness (706). Aluminum foil is preferably used for shielding the transparent surfaces from light.

When selecting the oocytes, a colored red-light filter is applied to the IVF workstation (Step 708). Lumar Decored SHRHPR red foil is the preferred colored red-light filter. Next, the cells are incubated in a light-protected place (Step 710).

The next in the process depends on whether IVF or ICIS is used (Step 712). If IVF is used, apply the colored red-light filter to protect the cells. If ICIS is used, apply the colored red-filter in combination with UV and IR shielding in the micromanipulation workstation (716).

What is claimed is:

1. A culture dish for protection to light, the culture dish comprises:
a. a base;
b. a wall connected to the outer rim of the base to form an enclosed base; and
c. a lid connected to the wall for creating a dark environment inside of the enclosed base.

2. The system of claim 1, where the base is circular.

3. The system of claim 1, where the base is made of polystyrene.

4. The system of claim 1, where a plurality of micro-wells is attached to the base.

5. The system of claim 1, where a plurality of interior walls is attached to the base.

6. The system of claim 1, where the lid is able to rotate on its axis, which is parallel to the axis of the base.

7. The system of claim 1, where the lid is made of aluminum foil with a thickness preferably ranging between 0.8 mm and 1.00 mm

8. A method for providing cells and embryos protect from light, the method comprises:
a. reducing room exposure to sunlight, where the cells and embryos are located;
b. covering the room ceiling lighting with color filter-luminaires;
c. shielding the transparent surfaces of the applied aspiration set and test tube from light;
d. applying a colored red-light filter to the IVF workstation; and
e. incubate cells and embryos in a light-protected place;

9. The method of claim 8, further comprising attaching a lid to a culture dish for creating a dark environment, which is absent of light, inside of the culture dish.

10. The method of claim 9, where the lid is made of aluminum foil with a thickness preferably ranging between 0.8 mm and 1.00 mm

11. The method of claim 8, where the colored red-light filter is applied to laboratory lamps and all instrument lighting;

12. The method of claim 8, where Lumar Decored SHRHPR red foil is the colored red-light filter.

13. The method of claim 8, further comprising using the color red-light filter to protect the cells and embryos in the IVF process.

14. The method of claim 8, further comprising using the color red-light filter in combination with ultra-violet (UV) and infra-red (IR) shielding in the micromanipulation workstation in the ICIS process.

15. The method of claim 11, where the UV and IR shielding is applied to microscopes.
